(19) United States
(12) Patent Application Publication
(10) Pub. No.: US 2025/0129141 A1
(43) Pub. Date: Apr. 24, 2025

(54) ANTIBODY FOR TETANUS TOXIN AND USE THEREOF

(71) Applicants: BEIJING WISDOMAB BIOTECHNOLOGY CO., LTD, Daxing District, Beijing (CN); GENRIX (SHANGHAI) BIOPHARMACEUTICAL CO. LTD., Pudong district, Shanghai (CN); CHONGQING GENRIX BIOPHARMACEUTICAL CO., LTD., Chongqing (CN)

(72) Inventors: Zhigang LIU, Beijing (CN); Xiaowei ZHOU, Beijing (CN); Yulan LIU, Beijing (CN); Xiaobo HAO, Beijing (AD); Junjie HU, Beijing (CN)

(73) Assignees: BEIJING WISDOMAB BIOTECHNOLOGY CO., LTD, Daxing District, Beijing (CN); GENRIX (SHANGHAI) BIOPHARMACEUTICAL CO. LTD., Pudong district, Shanghai (CN); CHONGQING GENRIX BIOPHARMACEUTICAL CO., LTD., Chongqing (CN)

(21) Appl. No.: 18/033,006

(22) PCT Filed: Nov. 19, 2020
(86) PCT No.: PCT/CN2020/130111

§ 371 Date: Apr. 20, 2023

(30) Foreign Application Priority Data
Oct. 21, 2020 (CN) 202011128926.0

(51) Int. Cl. C07K 16/12; A61K 39/00; A61P 39/02

(52) U.S. Cl. CPC C07K 16/1282; A61P 39/02; A61K 2039/505; C07K 2317/31; C07K 2317/55; C07K 2317/565; C07K 2317/622; C07K 2317/76; C07K 2317/92

(57) ABSTRACT

Provided are a bispecific antibody and a monoclonal antibody for a tetanus toxin, and uses of the antibodies. The bispecific antibody contains a first antigen-binding fragment and a second antigen-binding fragment which bind to different epitopes of the tetanus toxin, and has activity of neutralizing the tetanus toxin. The monoclonal antibody binds to the tetanus toxin, and has activity of neutralizing the tetanus toxin.

### CROSS REFERENCE TO RELATED APPLICATIONS

This application is a 35 U.S.C. § 371 National Phase Entry Application of International Application No. PCT/CN2020/130111 filed Nov. 19, 2020, which designates the U.S. and claims benefit under 35 U.S.C. § 119(a) of Chinese Patent Application No. 202011128926.0 filed on Oct. 21, 2020, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on May 12, 2023, is named 078291-000308USPX_SL.txt and is 56,081 bytes in size.

### TECHNICAL FIELD

The present application generally relates to the field of genetic engineering and antibody medicine. In particular, the present application relates to antibodies directed to a tetanus toxin and use thereof.

### BACKGROUND

Tetanus is an acute and fetal disease caused by neurotoxins secreted by Clostridium tetani, and attacks humans and animals. The most susceptible animals are horses, mules, or donkeys from the order Perssodactyla. Humans are also susceptible to neurotoxins secreted by Clostridium tetani. This disease can be observed at any age. Neonatal tetanus is one of the main causes of neonatal death in economically underdeveloped areas. The incubation period of tetanus is generally 3-21 days, mostly about 10 days, but it may also be one day to several months depending on the characteristics, extent and location of the wound. Typical clinical manifestations of tetanus are teeth clenching, increased stress of motor nerve center, and local or systemic muscle spasmodic paralysis. Patients often die of asphyxia and systemic organ failure. Even with modern intensive cares, the mortality rate in this disease is very high. The mortality rate of tetanus can be 19% to 31% especially after severe natural disasters. There are three clinical types of tetanus, i.e., systemic tetanus, local tetanus and head tetanus, which account for about 88%, about 12%, and about 1% resepetively in all cases.

Clostridium tetani is a strict anaerobic, gram-positive bacterium. It has flagella but no capsule. Its spores are widely found in fertilized soil, street dust, rancid sludge, animal intestines, and contaminated object surfaces. Clostridium tetani enters the human body mainly through a wound. If the wound is narrow and deep, or if the wound is simultaneously infected with aerobic pyogenic bacteria, Clostridium tetani will multiply in large number under anaerobic conditions and produce three exotoxins, among which the tetanus toxin causes characteristic symptoms of tetanus and elicits the production of protective antibodies [1]. However, about 20% of patients have no apparent invasive wounds, suggesting that Clostridium tetani can also invade the body through small scratch surfaces. Clostridium tetani can be divided into ten serotypes according to the agglutination reactions of flagellar antigen, but all serotypes produce the same neurotoxin. The toxins produced by individual types of strains can be neutralized by any type of antitoxin. The tetanus toxin is highly toxic, second only to botulinum toxin. Its lethal dose to mice is only 2-6 ng/kg.

The tetanus toxin, also known as the tetanus neurotoxin, is a single chain protein, which has a relative molecular weight of about 150 kDa, consists of 1315 amino acid residues, and can be cleaved into a light chain (fragment A, 50 kDa) and a heavy chain (HC, 100 kDa) linked via a disulfide bond, thereby forming its active form [2]. Fragment A is a zinc metalloproteinase that, upon entry into the cytoplasm, digests vesicle-associated membrane protein-2 (VAMP-2) and blocks the release of inhibitory neurotransmitters such as glycine and gamma-aminobutyric acid (GABA), resulting in muscle spasmodic paralysis [3]. The heavy chain comprises two functional domains. The C-terminal domain (Fragment C) binds to the surfaces of nerve cells such that the toxin molecule is endocytosed into vesicles. The N-terminal domain (Fragment B) passes the vesicle membrane and transports Fragment A into the neuronal cytoplasm [4]. Fragment C is subdivided into C-terminal subdomain (HCC) and N-terminal subdomain (HCN) [5]. The process by which the tetanus toxin enters neural cells is not fully understood. At present, a dual receptor mechanism is generally accepted, and involves a ganglioside receptor, in particular GT1b and GD1b, and a protein receptor [6].

Tetanus is a preventable disease. Subjects can receive active and passive immunization treatment after trauma. Humoral immunity has a protective effect on tetanus. Neutralizing antibodies bind to the toxin, and interfere with the interaction of the toxin with receptors on target cells and subsequent internalization of the toxin into the cell. Clinically, passive immunization is recommended for trauma patients who have unclear immunization history or are not immunized, have not received complete prime immunization, or have received complete prime immunization but with the last boost immunization received more than 5 years ago. Currently, there are two kinds of passive immune preparations for the prevention and treatment of tetanus in clinic. One is tetanus antitoxin (TAT), which is made from horse plasma immunized with tetanus toxoid by enzymatic digestion and salting-out. Before use of TAT, a skin test is required. TAT often causes allergic reactions with the incidence being 5%-30%. Anaphylactic shock occasionally occurs. The other kind of passive immune preparations is human immunoglobulin for tetanus (HIGT), which is made by taking plasma or serum with high titer of tetanus antibody from blood donors successively immunized with hepatitis B vaccine and tetanus toxoid, and processing the plasma or serum using the low temperature ethanol method. HIGT can be injected directly without skin test. However, as HIGT belongs to blood preparations, there is a potential risk of infectious diseases such as hepatitis C and AIDS. In addition, HIGT is limited by sources, and has low output, high cost and inconsistent quality among batches. [7]

Antibody drugs can overcome some of the above shortcomings and have certain advantages, such as production by stable expression by mammal cell expression systems, and definite composition.

Due to clinical needs, it is of great medical significance to develop antibodies that can neutralize the tetanus toxin and be used in preventing and treating tetanus.

### SUMMARY OF THE INVENTION

In a first aspect, there is provided in the present application a bispecific antibody comprising a first antigen-binding fragment and a second antigen-binding fragment that bind different epitopes of a tetanus toxin, wherein the bispecific antibody has activity of neutralizing the tetanus toxin.

In some embodiments of the first aspect, the first antigen-binding fragment comprises HCDR1 having the amino acid sequence of SYWIY (SEQ ID NO: 1), HCDR2 having the amino acid sequence of EINPTNGFANYNEKFKT (SEQ ID NO: 2) or EINPTAGFANYNEKFKT (SEQ ID NO: 3) or EINPTNAFANYNEKFKT (SEQ ID NO: 4), HCDR3 having the amino acid sequence of HFRFPY (SEQ ID NO: 5), LCDR1 having the amino acid sequence of RASQDIGSSLT (SEQ ID NO: 6), LCDR2 having the amino acid sequence of ATSSLDS (SEQ ID NO: 7) and LCDR3 having the amino acid sequence of LQYASSPYT (SEQ ID NO: 8); wherein the HCDR and LCDR amino acid sequences are defined according to Kabat.

In some embodiments of the first aspect, the second antigen-binding fragment comprises HCDR1 having the amino acid sequence of DYGVN (SEQ ID NO: 9), HCDR2 having the amino acid sequence of MIWSDGTTDYSSALKS (SEQ ID NO: 10), HCDR3 having the amino acid sequence of VDGYSHYYAMDY (SEQ ID NO: 11), LCDR1 having the amino acid sequence of RASENIYSYLA (SEQ ID NO: 12), LCDR2 having the amino acid sequence of NAKTLAE (SEQ ID NO: 13) and LCDR3 having the amino acid sequence of QHHYGLPFT (SEQ ID NO: 14); wherein the HCDR and LCDR amino acid sequences are defined according to Kabat.

In some embodiments of the first aspect, the amino acid sequence of the heavy chain variable region of the first antigen-binding fragment is as set forth in SEQ ID NO: 15, and the amino acid sequence of the light chain variable region of the first antigen-binding fragment is as set forth in SEQ ID NO: 18; or the amino acid sequence of the heavy chain variable region of the first antigen-binding fragment is as shown in SEQ ID NO: 16, and the amino acid sequence of the light chain variable region of the first antigen-binding fragment is as shown in SEQ ID NO: 18; or the amino acid sequence of the heavy chain variable region of the first antigen-binding fragment is as set forth in SEQ ID NO: 17, and the amino acid sequence of the light chain variable region of the first antigen-binding fragment is as set forth in SEQ ID NO: 18.

In some embodiments of the first aspect, the amino acid sequence of the heavy chain variable region of the second antigen-binding fragment is as set forth in SEQ ID NO: 19, and the amino acid sequence of the light chain variable region of the second antigen-binding fragment is as set forth in SEQ ID NO: 20.

In some embodiments of the first aspect, the forms of the first antigen-binding fragment and the second antigen-binding fragment are independently selected from a single-chain fragment variable (scFv) or a Fab fragment.

In a second aspect, there is provided in the application a monoclonal antibody that binds to a tetanus toxin, comprising:
HCDR1 having the amino acid sequence of SYWIY (SEQ ID NO: 1), HCDR2 having the amino acid sequence of EINPTNGFANYNEKFKT (SEQ ID NO: 2) or EINPTAGFANYNEKFKT (SEQ ID NO: 3) or EINPTNAFANYNEKFKT (SEQ ID NO: 4), HCDR3 having the amino acid sequence of HFRFPY (SEQ ID NO: 5), LCDR1 having the amino acid sequence of RASQDIGSSLT (SEQ ID NO: 6), LCDR2 having the amino acid sequence of ATSSLDS (SEQ ID NO: 7) and LCDR3 having the amino acid sequence of LQYASSPYT (SEQ ID NO: 8); wherein the HCDR and LCDR amino acid sequences are defined according to Kabat.

In a third aspect, there is provided in the application a monoclonal antibody that binds to a tetanus toxin, comprising:
HCDR1 having the amino acid sequence of DYGVN (SEQ ID NO: 9), HCDR2 having the amino acid sequence of MIWSDGTTDYSSALKS (SEQ ID NO: 10), HCDR3 having the amino acid sequence of VDGYSHYYAMDY (SEQ ID NO: 11), LCDR1 having the amino acid sequence of RASENIYSYLA (SEQ ID NO: 12), LCDR2 having the amino acid sequence of NAKTLAE (SEQ ID NO: 13), and LCDR3 having the amino acid sequence of QHHYGLPFT (SEQ ID NO: 14); wherein the HCDR and LCDR amino acid sequences are defined according to Kabat.

In a fourth aspect, there is provided in the application a pharmaceutical composition comprising the bispecific antibody of the first aspect, or the monoclonal antibody of the second or third aspect, and a pharmaceutically acceptable excipient, diluent, or carrier.

In a fifth aspect, there is provided in the application use of the bispecific antibody of the first aspect, the monoclonal antibody of the second or third aspect, or the pharmaceutical composition of the fourth aspect in the manufacture of a medicament for the prevention or treatment of tetanus.

### BRIEF DESCRIPTION OF THE DRAWINGS

FIG. 1 shows the results from an ELISA assay assessing how anti-TT-Hc monoclonal antibodies S22E2-mIgG2a and S24B8-mIgG2a inhibit binding of S22E2 purified phage to TT-Hc.

FIG. 2 shows the results from an ELISA assay assessing how anti-TT-Hc monoclonal antibodies S22E2-mIgG2a and S24B8-mIgG2a inhibit binding of S24B8 purified phage to TT-Hc.

FIG. 3 shows the results from an ELISA assay assessing how anti-TT-Hc monoclonal antibodies S22E2-mIgG2a and S24B8-mIgG2a inhibit binding of TT-Hc to ganglioside GT1b.

FIG. 4 shows the neutralization effects of the anti-tetanus toxin monoclonal antibodies on the tetanus toxin.

FIG. 5 shows the protective effect of the combination of anti-tetanus toxin monoclonal antibodies against lethal tetanus toxin attack in Balb/c mice.

FIG. 6 shows that the bispecific antibody S22E2h1-scFv-FcH1+S24B8h3-IgG1K inhibits binding of TT-Hc to ganglioside GM1.

FIG. 7 shows that the bispecific antibody S22E2h1-scFv-FcH1+S24B8h3-IgG1K inhibits binding of TT-Hc to ganglioside GD3.

FIG. 8 shows that the bispecific antibody S22E2h1-scFv-FcH1+S24B8h3-IgG1K inhibits binding of TT-Hc to ganglioside GT1b.

FIG. 9 shows the neutralizing activity of the anti-tetanus toxin bispecific antibody S22E2h1-scFv-FcH1+S24B8h3-IgG1K.

### DESCRIPTION OF THE SEQUENCES

SEQ ID NO: 1 shows the amino acid sequence of HCDR1 of the humanized heavy chain variable region mutants S24B8VH-h1, S24B8VH-h2 and S24B8VH-h3.

SEQ ID NO: 2 shows the amino acid sequence of HCDR2 of the humanized heavy chain variable region mutant S24B8VH-h1.

SEQ ID NO: 3 shows the amino acid sequence of HCDR2 of the humanized heavy chain variable region mutant S24B8VH-h2.

SEQ ID NO: 4 shows the amino acid sequence of HCDR2 of the humanized heavy chain variable region mutant S24B8VH-h3.

SEQ ID NO: 5 shows the amino acid sequence of HCDR3 of the humanized heavy chain variable region mutants S24B8VH-h1, S24B8VH-h2 and S24B8VH-h3.

SEQ ID NOs: 6-8 show the amino acid sequences of LCDR1, LCDR2 and LCDR3 of the humanized light chain variable region mutant S24B8VK-h1, respectively.

SEQ ID NOs: 9-11 show the amino acid sequences of HCDR1, HCDR2 and HCDR3 of the humanized heavy chain variable region mutant S22E2VH-h1, respectively.

SEQ ID NOs: 12-14 show the amino acid sequences of LCDR1, LCDR2 and LCDR3 of the humanized light chain variable region mutant S22E2VK-h1, respectively.

SEQ ID NO: 15 shows the amino acid sequence of the humanized heavy chain variable region mutant S24B8VH-h1.

SEQ ID NO: 16 shows the amino acid sequence of the humanized heavy chain variable region mutant S24B8VH-h2.

SEQ ID NO: 17 shows the amino acid sequence of the humanized heavy chain variable region mutant S24B8VH-h3.

SEQ ID NO: 18 shows the amino acid sequence of the humanized light chain variable region mutant S24B8VK-h1.

SEQ ID NO: 19 shows the amino acid sequence of the humanized heavy chain variable region mutant S22E2VH-h1.

SEQ ID NO: 20 shows the amino acid sequence of the humanized light chain variable region mutant S22E2VK-h1.

SEQ ID NO: 21 shows the amino acid sequence of S24B8VH-h1+CH-IgG1K.

SEQ ID NO: 22 shows the amino acid sequence of S24B8VH-h2+CH-IgG1K.

SEQ ID NO: 23 shows the amino acid sequence of S24B8VH-h3+CH-IgG1K.

SEQ ID NO: 24 shows the amino acid sequence of S24B8VK-h1+CK.

SEQ ID NO: 25 shows the amino acid sequence of S22E2-h1-scFv-FcH1.

SEQ ID NO: 26 shows the amino acid sequence of the C-terminal domain recombinant protein (TT-Hc) of the tetanus toxin heavy chain.

SEQ ID NO: 27 shows the amino acid sequence of the His tag.

SEQ ID NO: 28 shows the amino acid sequence of human (homo sapiens) IgG1 subtype heavy chain constant region.

SEQ ID NO: 29 shows the amino acid sequence of murine (mus musculus) IgG2a subtype heavy chain constant region.

SEQ ID NO: 30 shows the amino acid sequence of human IgG1 subtype mutant IgG1H.

SEQ ID NO: 31 shows the amino acid sequence of human IgG1 subtype mutant IgG1K.

SEQ ID NO: 32 shows the amino acid sequence of murine IgG2a subtype mutant mIgG2a-H.

SEQ ID NO: 33 shows the amino acid sequence of murine IgG2a subtype mutant mIgG2aK.

SEQ ID NO: 34 shows the amino acid sequence of human (homo sapiens) kappa subtype light chain constant region.

SEQ ID NO: 35 shows the amino acid sequence of human (homo sapiens) lamda subtype light chain constant region.

SEQ ID NO: 36 shows the amino acid sequence of murine (mus musculus) kappa subtype light chain constant region.

SEQ ID NO: 37 shows the amino acid sequence of murine (mus musculus) lamda subtype light chain constant region.

SEQ ID NO: 38 shows the nucleotide sequence of primer PmCGR.

SEQ ID NO: 39 shows the nucleotide sequence of primer PmCKR.

SEQ ID NO: 40 shows the amino acid sequence of the Fc segment containing the Knob mutation (FcK).

SEQ ID NO: 41 shows the amino acid sequence of the Fc segment containing the Hole mutation (FcH1).

### DETAILED DESCRIPTION OF THE INVENTION

The inventors of the present application conducted extensive research and development of antibody drugs against tetanus, and obtained novel bispecific antibodies and monoclonal antibodies against the tetanus toxin by antibody engineering techniques. The tetanus toxin is a macromolecular protein which consists of several domains. Theoretically, its has many epitopes that can elicits humoral immunity, and antibodies that bind to different epitopes can be obtained from immunized subjects. Fragment C of the tetanus toxin is a receptor-binding domain and it is likely that antibodies against Fragment C have neutralization activies by preventing the toxin from binding to gangliosides GT1b, GD1b and GM1, and achieving neutralizing effects on the toxin.

In various aspects of the present application, there are provided novel bispecific antibodies and mononal antibodies against the tetanus toxin, polynucleotides encoding the bispecific antibodies or mononal antibodies, vectors comprising the polynucleotides, host cells comprising the polynucleotides or vectors, methods of preparing and purifying the bispecific antibodies or mononal antibodies, and medical and biological use of the bispecific antibodies or mononal antibodies. Based on the sequences of the variable regions of the bispecific antibodies or mononal antibodies provided herein, full-length bispecific antibody molecules or mononal antibody molecules can be constructed for clinical use as a medicament for preventing or treating tetanus.

Unless otherwise indicated, the inventions of the present application can be practiced using conventional molecular biology, microbiology, cell biology, biochemistry, and immunological techniques in the art.

Unless otherwise indicated, the terms used in the present application have the meanings commonly understood by those skilled in the art.

### Definitions

As used herein, the term “antibody” refers to an immunoglobulin molecule that is capable of specifically binding to a target via at least one antigen recognition site located in a variable region of the immunoglobulin molecule. Targets include, but are not limited to, carbohydrates, polynucleotides, lipids, and polypeptides. As used herein, an “antibody” includes not only an intact (i.e., full-length) antibody, but also an antigen-binding fragment thereof (e.g., Fab, Fab′, F(ab′)₂, Fv), a variant thereof, a fusion protein comprising portions of an antibody, a humanized antibody, a chimeric antibody, a diabody, a linear antibody, a single-chain antibody, a multi-specific antibody (e.g., a bispecific antibody), and any other modified formats of an immunoglobulin molecule comprising a desired specific antigen recognition site, including a glycosylated variant of an antibody, an amino acid sequence variant of an antibody, and a covalently modified antibody.

Typically, an intact or full-length antibody comprises two heavy chains and two light chains. Each heavy chain contains a heavy chain variable region (VH) and first, second and third constant regions (CH1, CH2 and CH3). Each light chain contains a light chain variable region (VL) and a constant region (CL). A full-length antibody may be of any type, such as an IgD, IgE, IgG, IgA, or IgM (or their subtypes) antibody, but not necessarily belong to any particular type. Immunoglobulins can be assigned to different types depending on their amino acid sequences of the heavy chain constant domains. Generally, immunoglobulins have five main types, i.e., IgA, IgD, IgE, IgG, and IgM, and some of these types can be further classified into subtypes (isotypes), such as IgG1, IgG2, IgG3, IgG4, IgA1, and IgA2. Heavy chain constant domains corresponding to individual immunoglobulin types are referred to as α, δ, ε, γ, and μ, respectively. Subunit structures and three-dimensional structures of different types of immunoglobulins are well known.

As used herein, the term “bispecific antibody” is an antibody having the ability to bind to two different antigens. A bispecific antibody can have a variety of structural configurations. For example, a bispecific antibody may consist of two Fc fragments and two antigen-binding portions fused thereto, respectively (similar to a natural antibody, except that the two arms bind to different antigen targets or epitopes). Antigen-binding portions can be in the form of scfv or Fab fragments. When two antigens are given, two different antigen-binding portions of a bispecific antibody each binds to the N-terminus of one Fc fragment. The configuration of the antigen-binding portions of the two arms can have the four combinations, i.e., scfv+Fab fragment, Fab fragment +scfv, scfv+scfv, and Fab fragment+Fab fragment. Fc fragments can contain mutations that can ensure heavy chain heteromerization. The KIH (knob-in-hole) technique is one strategy to address heavy chain heteromerization. Generally, the KIH technique refers to formation of a structure that facilitates the pairing of heterogeneous hemibodies by engineering the amino acid sequence of a CH3 region. This can maintain the structure of a normal antibody as much as possible while forming a bispecific antibody. For guidance of the KIH technique, see, for example, “An efficient route to human bispecific IgG”, A. Margaret Merchant et al., Nature Biotechnology, Volume 16, 1998, which is incorporated herein by reference in its entirety. Furthermore, a bispecific antibody can be configured in a way that an antibody that binds to a first antigen (e.g., in the form of a natural antibody) has an antigen-binding portion that can bind to a second antigen extending from the C-terminus of the CH₃ region (e.g., via a flexible linker).

As used herein, the term “antigen-binding portion” or “antigen-binding fragment” can be used interchangeably, and refers to a portion or region of an intact antibody molecule responsible for binding to an antigen. An antigen binding domain can comprise a heavy chain variable region (VH), a light chain variable region (VL), or both. Each of a VH and a VL typically contains three complementarity determining regions, i.e., CDR1, CDR2, and CDR3.

It is well known to those skilled in the art that complementarity determining regions (CDRs, usually including CDR1, CDR2 and CDR3) are the regions of a variable region that have mostly impact on the affinity and specificity of an antibody. The CDR sequences of a VH or VL have two common definitions, i.e., the Kabat definition and the Chothia definition (see, e.g., Kabat, “Sequences of Proteins of Immunological Interest”, National Institutes of Health, Bethesda, Md.(1991); A1-Lazikani et al., J. Mol. Biol. 273:927-948 (1997); and Martin et al., Proc. Natl. Acad. Sci. USA 86: 9268-9272 (1989)). For the variable region sequences of a given antibody, the sequences of CDR regions in the VH and VL can be determined according to the Kabat definition or the Chothia definition. In an embodiment of the present application, CDR sequences are defined according to Kabat.

For the variable region sequences of a given antibody, the sequences of CDR regions in the variable region sequences can be analyzed in a variety of ways, for example, using online software Abysis (http://www.abysis.org/).

For conventional antibodies, examples of an antigen-binding fragment include, but are not limited to, (1) an Fab fragment, which can be a monovalent fragment having a VL-CL chain and a VH-CH1 chain; (2) an F(ab′)₂ fragment, which can be a divalent fragment having two Fab′ fragments linked by a disulfide bridge of the hinge region (i.e., a dimer of Fab′); (3) an Fv fragment having VL and VH domains in a single arm of an antibody; (4) a single chain Fv (scFv), which can be a single polypeptide chain consisting of a VH domain and a VL domain via a polypeptide linker; and (5) (scFv)₂, which can comprise two VH domains linked by a peptide linker and two VL domains that are combined with the two VH domains via a disulfide bridge.

In bispecific antibody construction, an “antigen-binding portion” includes, but is not limited to, a Fab fragment or a single-chain fragment variable (scFv).

As used herein, the term “single chain fragment variable (scFv)” refers to an antibody of a single chain structure generally constructed using genetic engineering techniques, comprising a polypeptide chain comprising a heavy chain variable region (VH) and a light chain variable region (VL). A flexible linker is typically designed between the heavy chain variable region and the light chain variable region so that the heavy chain variable region and the light chain variable region can be folded into the correct conformation for binding to an antigen.

As used herein, the term “Fab (fragment antigen-binding) fragment”, “Fab portion”, or the like refers to an antibody fragment that is produced after treatment of an intact antibody with papain and is capable of binding to an antigen, including the intact light chain (VL-CL), the heavy chain variable region, and the CH1 fragment (VH-CH1).

As used herein, the term “monoclonal antibody” refers to an antibody from a substantially homogeneous antibody population, which means that the antibodies constituting the population are the same except for naturally occurring mutations which may occur in a small number of individual antibodies. Monoclonal antibodies described herein particularly include “chimeric” antibodies in which a portion of the heavy and/or light chain is identical or homologous to a corresponding sequence in an antibody derived from a particular species or belonging to a particular antibody type or subtype, while the remainder of the heavy and/or light chain is identical or homologous to a corresponding sequence in an antibody derived from another species or belonging to another antibody type or subtype, and also include fragments of such antibodies as long as they exhibit desired biological activity (see, U.S. Pat. No. 4,816,567; and Morrison et al., Proc. Natl. Acad. Sci. USA 81: 6851-6855 (1984)).

As used herein, the term “specific binding” refers to a non-random binding reaction between two molecules, e.g., binding of an antibody to an antigen epitope.

In a first aspect, there is provided in the present application a bispecific antibody comprising a first antigen-binding fragment and a second antigen-binding fragment that bind to different epitopes of a tetanus toxin, wherein the bispecific antibody has activity of neutralizing the tetanus toxin.

In some embodiments of the first aspect, the first antigen-binding fragment comprises HCDR1 having the amino acid sequence of SYWIY (SEQ ID NO: 1), HCDR2 having the amino acid sequence of EINPTNGFANYNEKFKT (SEQ ID NO: 2) or EINPTAGFANYNEKFKT (SEQ ID NO: 3) or EINPTNAFANYNEKFKT (SEQ ID NO: 4), HCDR3 having the amino acid sequence of HFRFPY (SEQ ID NO: 5), LCDR1 having the amino acid sequence of RASQDIGSSLT (SEQ ID NO: 6), LCDR2 having the amino acid sequence of ATSSLDS (SEQ ID NO: 7) and LCDR3 having the amino acid sequence of LQYASSPYT (SEQ ID NO: 8); wherein the HCDR and LCDR amino acid sequences are defined according to Kabat.

In some embodiments of the first aspect, the second antigen-binding fragment comprises HCDR1 having the amino acid sequence of DYGVN (SEQ ID NO: 9), HCDR2 having the amino acid sequence of MIWSDGTTDYSSALKS (SEQ ID NO: 10), HCDR3 having the amino acid sequence of VDGYSHYYAMDY (SEQ ID NO: 11), LCDR1 having the amino acid sequence of RASENIYSYLA (SEQ ID NO: 12), LCDR2 having the amino acid sequence of NAKTLAE (SEQ ID NO: 13) and LCDR3 having the amino acid sequence of QHHYGLPFT (SEQ ID NO: 14); wherein the HCDR and LCDR amino acid sequences are defined according to Kabat.

In some embodiments of the first aspect, the amino acid sequence of the heavy chain variable region of the first antigen-binding fragment is as set forth in SEQ ID NO: 15, and the amino acid sequence of the light chain variable region is as set forth in SEQ ID NO: 18; or the amino acid sequence of the heavy chain variable region of the first antigen-binding fragment is as shown in SEQ ID NO: 16, and the amino acid sequence of the light chain variable region of the first antigen-binding fragment is as shown in SEQ ID NO: 18; or the amino acid sequence of the heavy chain variable region of the first antigen-binding fragment is as set forth in SEQ ID NO: 17, and the amino acid sequence of the light chain variable region of the first antigen-binding fragment is as set forth in SEQ ID NO: 18.

In some embodiments of the first aspect, the amino acid sequence of the heavy chain variable region of the second antigen-binding fragment is as set forth in SEQ ID NO: 19, and the amino acid sequence of the light chain variable region of the second antigen-binding fragment is as set forth in SEQ ID NO: 20.

In some embodiments of the first aspect, the forms of the first antigen-binding fragment and the second antigen-binding fragment are independently selected from a single-chain fragment variable (scFv) or a Fab fragment.

In some embodiments of the first aspect, the first antigen-binding fragment is a Fab fragment, and the second antigen-binding fragment is a single-chain fragment variable (scFv).

In some embodiments of the first aspect, the bispecific antibody comprises the amino acid sequence set forth in one of SEQ ID NOs: 21, 22, and 23 and the amino acid sequence set forth in SEQ ID NO: 24.

In some embodiments of the first aspect, the bispecific antibody comprises the amino acid sequence set forth in SEQ ID NO: 25.

In a second aspect, there is provided in the application a monoclonal antibody that binds to a tetanus toxin, comprising:
HCDR1 having the amino acid sequence of SYWIY (SEQ ID NO: 1), HCDR2 having the amino acid sequence of EINPTNGFANYNEKFKT (SEQ ID NO: 2) or EINPTAGFANYNEKFKT (SEQ ID NO: 3) or EINPTNAFANYNEKFKT (SEQ ID NO: 4), HCDR3 having the amino acid sequence of HFRFPY (SEQ ID NO: 5), LCDR1 having the amino acid sequence of RASQDIGSSLT (SEQ ID NO: 6), LCDR2 having the amino acid sequence of ATSSLDS (SEQ ID NO: 7) and LCDR3 having the amino acid sequence of LQYASSPYT (SEQ ID NO: 8); wherein the HCDR and LCDR amino acid sequences are defined according to Kabat.

In some embodiments of the second aspect, the monoclonal antibody comprises a heavy chain variable region amino acid sequence set forth in SEQ ID NO: 15, and a light chain variable region amino acid sequence set forth in SEQ ID NO: 18; or a heavy chain variable region amino acid sequence set forth in SEQ ID NO: 16, and a light chain variable region amino acid sequence set forth in SEQ ID NO: 18; or a heavy chain variable region amino acid sequence set forth in SEQ ID NO: 17, and a light chain variable region amino acid sequence set forth in SEQ ID NO: 18.

In a third aspect, there is provided in the application a monoclonal antibody that binds to a tetanus toxin, comprising:
HCDR1 having the amino acid sequence of DYGVN (SEQ ID NO: 9), HCDR2 having the amino acid sequence of MIWSDGTTDYSSALKS (SEQ ID NO: 10), HCDR3 having the amino acid sequence of VDGYSHYYAMDY (SEQ ID NO: 11), LCDR1 having the amino acid sequence of RASENIYSYLA (SEQ ID NO: 12), LCDR2 having the amino acid sequence of NAKTLAE (SEQ ID NO: 13), and LCDR3 having the amino acid sequence of QHHYGLPFT (SEQ ID NO: 14); wherein the amino acid sequences of HCDR and LCDR are defined according to Kabat.

In some embodiments of the third aspect, the monoclonal antibody comprises a heavy chain variable region amino acid sequence set forth in SEQ ID NO: 19, and a light chain variable region amino acid sequence set forth in SEQ ID NO: 20.

In a fourth aspect, there is provided in the application a pharmaceutical composition comprising the bispecific antibody of the first aspect, or the monoclonal antibody of the second or third aspect, and a pharmaceutically acceptable excipient, diluent, or carrier.

In some embodiments of the fourth aspect, the pharmaceutical composition is for preventing or treating tetanus.

In some embodiments of the fourth aspect, the pharmaceutical composition may further comprise one or more of a lubricant, such as talc, magnesium stearate, and mineral oil; a wetting agent; an emulsifier; a suspending agent; a preservative such as benzoic acid, sorbic acid and calcium propionate; a sweetening agent and/or a flavoring agent.

In some embodiments of the fourth aspect, the pharmaceutical composition herein may be formulated as a tablet, a pill, a powder, a lozenge, an elixir, a suspension, an emulsion, a solution, a syrup, a suppository, or a capsule.

In some embodiments of the fourth aspect, the pharmaceutical composition of the present application may be delivered using any physiologically acceptable administration route including, but not limited to, oral administration, parenteral administration, nasal administration, rectal administration, intraperitoneal administration, intravascular injection, subcutaneous administration, transdermal administration, or inhalation administration.

In some embodiments of the fourth aspect, a pharmaceutical composition for therapeutic use may be formulated for storage in a lyophilized formulation or in the form of an aqueous solution by mixing an agent with desired purity with a pharmaceutically acceptable carrier or excipient where appropriate.

In a fifth aspect, there is provided in the application use of the bispecific antibody of the first aspect, the monoclonal antibody of the second or third aspect, or the pharmaceutical composition of the fourth aspect in the manufacture of a medicament for the prevention or treatment of tetanus.

In a sixth aspect, there is provided in the application a method of preventing or treating tetanus, comprising administering to a subject in need thereof the bispecific antibody of the first aspect, the monoclonal antibody of the second or third aspect, or the pharmaceutical composition of the fourth aspect.

In other aspects, there is provided in the application a nucleic acid molecule encoding the bispecific antibody of the first aspect, or the monoclonal antibody of the second or third aspect. In some embodiments, the nucleic acid molecule is operably linked to a regulatory sequence that can be recognized by a host cell transformed with the vector.

There is also provided in the application a vector comprising an isolated nucleic acid molecule encoding the bispecific antibody of the first aspect, or the monoclonal antibody of the second or third aspect, and a host cell comprising the nucleic acid molecule or the vector.

In other aspects, there is provided in the application a method of producing the bispecific antibody of the first aspect, or the monoclonal antibody of the second or third aspect. In some embodiments, the method of producing the bispecific antibody of the first aspect, or the monoclonal antibody of the second or third aspect comprises culturing a host cell so as to facilitate expression of the nucleic acid. In some embodiments, the method of producing the bispecific antibody of the first aspect, or the monoclonal antibody of the second or third aspect further comprises recovering the bispecific antibody or monoclonal antibody from the host cell culture medium.

It is to be understood that the foregoing detailed description is intended only to enable those skilled in the art to have better understanding of the present application and is not intended to cause limitations in any way. Various modifications and variations can be made to the described embodiments by those skilled in the art.

The following Examples are for purposes of illustration only and are not intended to limit the scope of the present application.

### EXAMPLES

### Example 1: Preparation of Recombinant Tetanus Toxin Antigen and Recombinant Antibodies

### 1.1 Preparation of Recombinant Tetanus Toxin Antigen

A recombinant protein of C-terminal domain of tetanus toxin heavy chain (TT-Hc, SEQ ID NO:26) was used in the production of monoclonal antibodies against tetanus toxin. This protein was derived from Clostridium tetani and was lack of any post-translational modification, and therefore can be expressed using an E. coli expression system. Furthermore, an His tag (His, SEQ ID NO:27) was added to the C-terminus of the recombinant protein, so as to facilitate the purification of the recombinant protein and the identification of the function of the monoclonal antibody.

A gene encoding TT-Hc (containing the His tag) was designed and synthesized according to the amino acid sequences of the TT-Hc recombinant proteins in the Uniprot database. The synthesized gene was cloned into a suitable eukaryotic expression vector (such as pET-22b, Invitrogen, Inc.) using conventional molecular biology techniques. Expression plasmids were transformed into competent E. coli cells (such as BL21 (DE3), Solarbio, Inc.). The E. coli expressing the TT-Hc-His recombinant protein was induced to express by IPTG (such as 18070-1g, Solarbio, Inc.). Then, the bacteria were collected by centrifugation and disrupted by an ultrasonic cell crusher (such as VCX130, Sonics, Inc.). The supernatants were harvested by centrifugation. The recombinant protein in the supernatants was further purified by a metal-chelated affinity chromatography column (such as HisTrap FF, GE, Inc.). The recombinant protein preservation buffer was then replaced with PBS (pH 7.0) or other suitable buffers by using a desalting column (such as Hitrap desaulting, GE, Inc.). If necessary, the sample can be sterilized by filtration and then stored in aliquots at −20° C.

### 1.2. Preparation of Recombinant Antibodies

Nucleotide sequences encoding the antibody heavy chain variable region and light chain variable region were cloned into eukaryotic expression vectors (such as pcDNA3.1, Invitrogen, Inc.) carrying the nucleotide sequences encoding the heavy chain constant region and the light chain constant region, respectively, using conventional molecular biological means. Intact antibodies were expressed in combination. The heavy chain constant regions of antibodies can be of human IgG1 subtype (SEQ ID NO: 28), or murine IgG2a subtype (SEQ ID NO: 29), or can be human IgG1 subtype mutant IgG1H (SEQ ID NO: 30) and IgG1K (SEQ ID NO: 31) based on the KIH (Knob-Into-Hole) technology, or can be murine IgG2a subtype mutant mIgG2a-H (SEQ ID NO: 32) and mIgG2aK (SEQ ID NO: 33). The light chain constant regions can be of human kappa subtype (SEQ ID NO: 34), human lambda subtype (SEQ ID NO: 35), murine kappa subtype (SEQ ID NO: 36) or murine lambda subtype (SEQ ID NO: 37).

The prepared recombinant antibody expression plasmids were transfected into HEK293 cells (such as HEK293F, Invitrogen, Inc.) using liposomes (such as 293fectin, Invitrogen, Inc.) or other transfected reagents (such as PEI). The cells were cultured in suspension in serum-free mediums for 3-5 days. The culture supernatants were then harvested by centrifugation. The recombinant protein in the supernatant was further purified by a ProteinA/G affinity chromatography column (such as Mabselect SURE, GE, Inc.). The recombinant protein preservation buffer was then replaced with PBS (pH 7.0) or other suitable buffers by using a desalting column (such as Hitrap desaulting, GE, Inc.). If necessary, the antibody sample can be sterilized by filtration and then stored in aliquots at −20° C.

### Example 2: Murien Immunization and Construction of Immune Library

BALB/c mice with 6 to 8 weeks old were immunized with the recombinant protein TT-Hc-His prepared in Example 1 as the antigen at an immunization dose of 50 μg per mouse. Boosted immunization was carried out every 14 days. The mice were sacrificed 8 weeks after prime immunization and spleen cells were harvested. Murine spleen lymphocytes were isolated by using a murine lymphocyte isolation solution (Dakewe Biotech Co., Ltd., CAT #DKW33-R0100). Total RNA was extracted from the isolated lymphocytes by using a cellular total RNA extraction kit (Tiangen Biotech (Beijing) Co., Ltd., CAT #DP430). Using the extracted total RNA as a template, the antibody heavy chain variable region and light chain variable region were synthesized using a first strand cDNA synthesis kit (Thermo scientific, CAT #K1621). The used reverse transcription primers were gene-specific primers, and the primer pairing regions were located in the heavy chain constant region and the antibody light chain constant region, respectively. The specific sequences were PmCGR: TGCATTTGAACTCCTTGCC (SEQ ID NO: 38) and PmCKR: CCATCAATCTTCCACTTGAC (SEQ ID NO: 39), respectively. The synthesized cDNA was immediately stored at −70° C. for subsequence use. Then, using the cDNA obtained by reverse transcription as a template, the primers were synthesized by referring to Krebber A, Bornhauser S, Burmester J, et al. Reliable cloning of functional antibody variable domains from hybridomas and spleen cell repertoires employing a reengineered phage display system. J Immunol Methods. 1997;201(1):35-55, the entire contents of which are incorporated herein by reference, and the nucleotide sequences encoding the VH and VK of murine antibodies were respectively amplified by PCR. Then, the single-chain fragment variable (scFv) genes were constructed by overlapping extension PCR technology. Finally, the nucleotide sequences of the prepared murine single chain antibodies were cloned into the vector pADSCFV-S (see Example 1 in Chinese Patent Application No. 201510097117.0 for detailed experimental protocols, the entire contents of which are incorporated herein by reference) to construct a scFv library. The antibody library has a capacity of 5×10E8, and an accuracy of 50%.

### Example 3: Screening and Preliminary Identification of Murine Immune Library

### 3.1. Screening of Murine Immune Library

Referring to Chinese Patent Application No. 201510097117.0 (the entire contents of which are incorporated herein by reference), the phage library displaying murine single chain antibodies constructed in Example 2 was screened by solid phase screening strategy (as for the experimental protocal, see, Phage Display: A Practical Approach, Tim Clackson and Henry B. Lowman (ed.), translated by Lan Ma et al., Chemical Industry Press, May 2008) using the recombinant human TT-Hc-His prepared in Example 1 as the antigen. Three rounds of screening were carried out by means of binding, elution, neutralization, infection and amplification. Finally, two single chain antibodies S22E2 and S24B8 specifically binding to human TT-Hc-His were obtained.

The nucleotide sequences encoding the heavy chain and light chains of S22E2 and S24B8 were respectively cloned into eukaryotic expression vectors carrying the nucleotide sequences encoding the mouse mIgG2a heavy chain constant region and the mouse kappa light chain constant region by using conventional molecular biological means so as to prepare the recombinant murine antibodies.

### 3.2. Affinity Analysis of Recombinant Anti-TT-Hc Monoclonal Antibodies

The affinities of the anti-TT-Hc antibodies were determined by the surface plasmon resonance technique using Biacore X100. Reagents and consumables such as amino-coupling kit (BR-1000-50), murine antibody capture kit (BR-1008-38), CM5 chip (BR100012) and 10×HBS-EP, pH 7.4 (BR100669) were purchased from GE healthcare. The carboxylated CM5 chip surface was activated with 1-Ethyl-3-(3-dimethylaminopropyl) carbodiimide hydrochlorid (EDC) and N-Hydroxysuccinimide (NHS) according to the instructions in the kit. An anti-mouse IgG (Fc) antibody (capture antibody) was diluted to 25 μg/mL with 10 mM sodium acetate (pH5.0), and then injected at a flow rate of 10 μL/min to achieve a coupling amount of approximately up to 10,000 response units (RU). After injection of the capture antibody, 1 M ethanolamine was injected to block unreacted groups. For the kinetics measurement, the anti-TT-Hc antibody was diluted to 0.5-1 μg/mL, and injected at 10 μL/min to ensure that approximately 50RU of the antibody was captured by the anti-murine Fc antibody. The TT-Hc-His was then set to a series of concentration gradients (e.g., 6.17 nM, 18.5 nM, 55.6 nM, 167 nM, and 500 nM) and injected at 25° C. from lower to higher concentrations at 30 μL/min, with a association time of 120 s and a dissociation time of 3,600 s. A 10 mM glycine-HCl solution (pH2.0) was injected at 10 μL/min for 30 s to regenerate the surface of the chip. The association rate (Ka) and dissociation rate (Kd) were calculated by fitting the binding and dissociation sensorgrams with a 1:1 binding model by using Biacore X100 evaluation software version 2.0.1. The dissociation equilibrium constant (KD) was calculated as the ratio K_{d}/Kₐ. The fitting results were shown in Table 1.

TABLE 1
Affinity constant in binding of recombinant
anti-TT-Hc monoclonal antibodies to TT-Hc-his

| | Kₐ | K_{d} | KD |
|---|---|---|---|
| S22E2-mIgG2a | 6.424E+4 | 1.589E−4 | 2.473E−9 |
| S24B8-mIgG2a | 5.488E+4 | 1.015E−5 | 1.849E−10 |

### Example 4: Epitope Analysis of Anti-Tetanus Toxin Monoclonal Antibodies

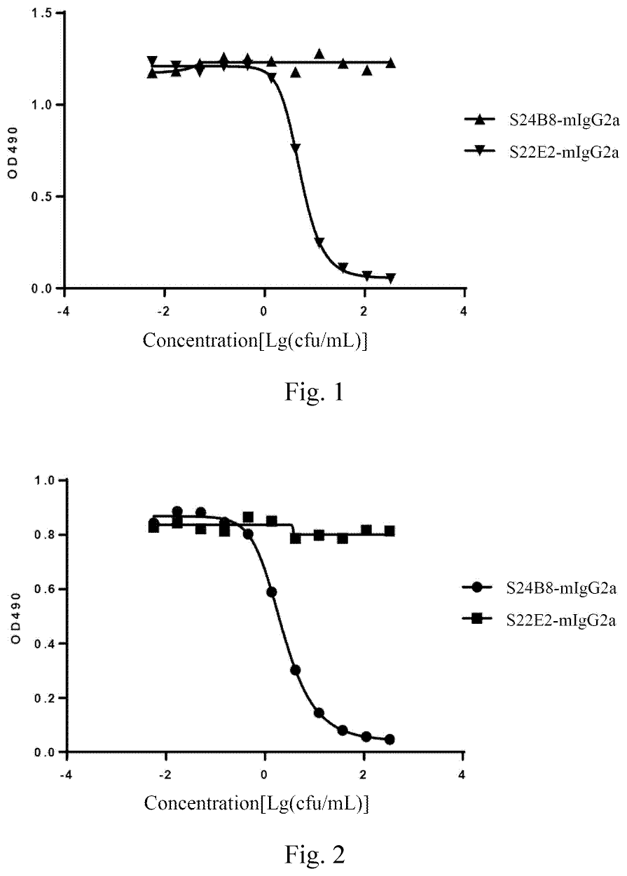

96-well ELISA plates were coated with recombinant protein TT-Hc-His (3 μg/mL, 100 μL/well) overnight in a refrigerator at 4° C., and were then blocked with a blocking solution (PBS-0.1% Tween20-3% milk) at 37° C. for 1 hour. The S22E2-mIgG2a and S24B8-mIgG2a murine antibodies were diluted gradiently with S22E2 purified phage (S22E2 phage) of a fixed concentration (1*10¹¹ cfu/mL). The starting concentration of the antibodies was 50 μg/mL, and the dilution was carried out at 3-fold for a total of 11 concentration gradients. The antibody dilutions were added to the blocked 96-well ELISA plates at 100 μL/well and the plates were incubated at 37° C. for 1 hour. Then, the ELISA plates were washed with PBS-0.1% Tween20, followed by addition of HRP anti-M13 secondary antibody (Sino Biological Inc., 11973-MM05T-H) and the plates were incubated at 37° C. for 1 hour. The ELISA plates were washed with PBS-0.1% Tween20, and an OPD substrate developing solution was added. After incubation for 5-10 minutes, the development was terminated with 1M H₂SO₄. The optical density values were determined using a microplate reader at 492 nm/630 nm dual wavelength. The results from the ELISA assay (FIG. 1) showed that S22E2-mIgG2a completely inhibited the binding signal between S22E2 phage and recombinant protein TT-Hc, but S24B8-mIgG2a had no effect on binding of S22E2 phage to TT-Hc.

Similarly, S22E2-mIgG2a and S24B8-mIgG2a were diluted gradiently with S24B8 purified phage (S24B8 phage) of the fixed concentration. The binding signal between S24B8 phage and TT-Hc was detected with HRP anti-M13 secondary antibody. The ELISA results (FIG. 2) showed that S24B8-mIgG2a completely inhibited the binding signal between S24B8 phage and recombinant protein TT-Hc, but S22E2-mIgG2a has little effect on binding of S24B8 phage to TT-Hc.

In summary, anti-TT-Hc monoclonal antibodies S22E2-mIgG2a and S24B8-mIgG2a bind to different epitopes of TT-Hc.

### Example 5: Inhibition of Binding of TT-Hc to Gangliosides by Anti-Tetanus Toxin Monoclonal Antibodies

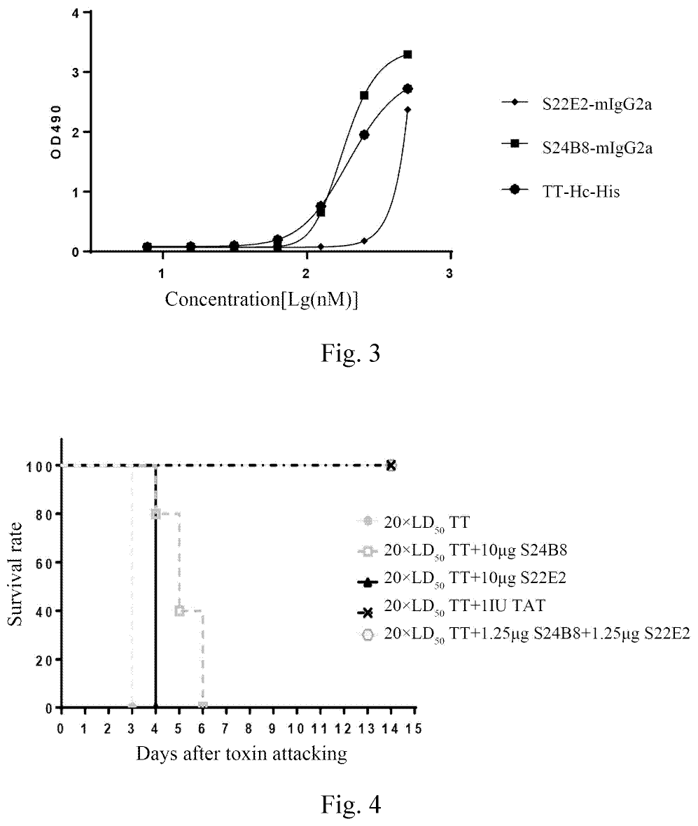

The tetanus toxin can bind to specific receptors on the surfaces of nerve cells via the Hc domain. Currently, the generally accepted mechanism is a dual receptor mechanism, involving a ganglioside receptor and a protein receptor. 96-well ELISA plates were coated with gangliosides GT1b (100 μL/well) which was diluted to 10 μg/mL with methanol, and the plates were left overnight at room temperature to allow the methanol to evaporate. Then, the plates were blocked with a blocking solution (PBS-0.1% Tween20-1% BSA) at 37° C. for 1 hour. TT-Hc was diluted gradiently. The starting concentration of TT-Hc was 100 μg/mL and the dilution was carried out at 3-fold for a total of 7 concentration gradients. The anti-TT-Hc murine monoclonal antibodies were diluted to 40 μg/mL with PBS and mixed with TT-Hc dilutions in equal volumes, and then the mixed solutions were added to the blocked 96-well ELISA plates at 100 μL/well. A TT-Hc control group without an antibody was set. The plates were incubated at 37° C. for 1 hour. The ELISA plates were washed with PBS-0.1% Tween20, and a HRP anti-TT polyclonal antibody (prepared in laboratory) was added at 100 μL/well. The plates were incubated at 37° C. for 1 hour. Then, the ELISA plates were washed with PBS-0.1% Tween20, and an OPD substrate developing solution was added. After incubation for 5-10 minutes, the development was terminated with 1 M H₂SO₄ solution, and the optical density values were determined using a microplate reader at 492 nm/630 nm dual wavelength. The results from the ELISA assay (FIG. 3) showed that, with respect to the binding curve of TT-Hc as the basis, the binding curve significantly shifted to the right after the addition of S22E2-mIgG2a, indicating that S22E2-mIgG2a effectively inhibited binding of TT-Hc to ganglioside GT1b. In contrast, S24B8-mIgG2a did not inhibit binding of TT-Hc to ganglioside, and instead showed somewhat promoting effects.

### Example 6: Neutralizing Activities of Anti-Tetanus Toxin Monoclonal Antibodies

The neutralizing activity of an antibody was determined by mixing the antibody and a lethal dose of tetanus toxin in vitro and then injecting the mixture into Balb/c mice. In particular, the tetanus toxin was mixed with either an anti-tetanus toxin monoclonal antibody or an antitoxin, and the mixture was left at 37° C. for 1 hour and then injected into the muscle of right hind legs of Balb/c mice. The injection dose of tetanus toxin was 20×LD₅₀/mouse. The injection dose of commercially available equine tetanus immunoglobulin (F(ab′)₂) (labeled as TAT, Shanghai Serum Bio-technology Co., LTD.) was 1 IU/mouse. The injection dose of monoclonal antibody S22E2 or S24B8 was 10 μg/mouse. The dose of S24B8 and S22E2 in combination was 1.25 μg/mouse for each antibody. Balb/c mice injected with 20×LD₅₀ of tetanus toxin were used as a control group. There were a total of 5 groups each of which had 8 mice. The tetanus toxin was diluted to 800×LD₅₀/mL with a boric acid buffer (i.e., 20×LD₅₀ per 50 μl of injection after mixing with the antibody in equal volumes). Equine tetanus immunoglobulin was diluted to 40 IU/mL with a boric acid buffer (i.e., 1 IU per 50 μl of injection after mixing with the tetanus toxin in equal volumes). Antibodies S22E2 and S24B8 were diluted to 0.4 mg/ml (or 0.1 mg/mL) with a boric acid buffer (i.e., for the treatment with S22E2 or S24B8, 10 μg of an antibody per 50 μl of injection after mixing with the tetanus toxin in equal volumes; and for the treatment with S24B8 and S22E2 in combination, 1.25 μg of each antibody per 50 μl of injection after mixing the two-antibody mixture (mixed in equal volumes) with the tetanus toxin in equal volumes). Each animal was injected with 50 μL of the mixture of the tetanus toxin and an antibody. The experimental results were shown in FIG. 4. All Balb/c mice in the control group died of tetanus within 3 days of the attack. Under the experimental conditions, monoclonal antibody S24B8 or S22E2 partially neutralized the toxin and prolonged the survival of the mice. In contrast, the combination of S24B8 and S22E2 completely neutralized the toxin and all mice survived by the end of the experimental period, same as the neutralizing activity observed for the equine tetanus immunoglobulin TAT.

### Example 7: Protection from Combination of Anti-Tetanus Toxin Monoclonal Antibodies in Balb/c Mice Receiving Lethal Tetanus Toxin Attack

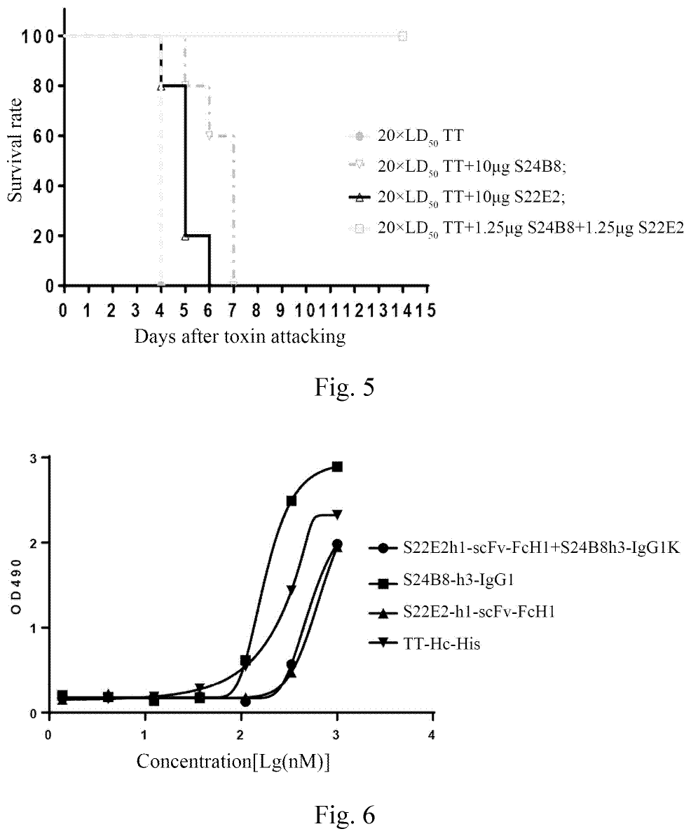

Balb/c mice were subjected to immunization prior to immunoglobulin exposure and then the mice injected with an antibody were attacked with a lethal dose of tetanus toxin to determine the protective effects of the antibody. The injection dose of monoclonal antibodies S22E2 or S24B8 was 10 μg/mouse. The injection dose of S24B8 and S22E2 in combination was 1.25 μg/mouse for each antibody. Balb/c mice injected with 20×LD₅₀ of tetanus toxin were used as a control group. There were a total of 4 groups each of which had 8 mice. The tetanus toxin was diluted to 400×LD₅₀/mL with a boric acid buffer (i.e., containing 20×LD₅₀ per 50 μl of injection). Antibodies S22E2 and S24B8 were diluted to 0.2 mg/mL (or 0.05 mg/mL) with a boric acid buffer (i.e., for the treatment with S22E2 or S24B8, 10 μg of an antibody per 50 μl of injection; and for the treatment with S24B8 and S22E2 in combination, 1.25 μg of each antibody per 50 μl of injection after the two antibodies were mixed in equal volumes. 50 μL of anti-tetanus toxin antibodies were administered to Balb/c mice, and 24 hours later, the mice were attacked with the tetanus toxin in the muscle of right hind legs. Each animal was injcted with 50 μL of tetanus toxin. The experimental results were shown in FIG. 5. All Balb/c mice in the control group died of tetanus within 4 days of the attack. Under the experimental conditions, the combination of S24B8 and S22E2 completely protected Balb/c mice, all of which survived by the end of the experimental period.

### Example 8: Humanization and Deamination Site Mutation of Anti-Tetanus Toxin Monoclonal Antibodies

### 8.1. Humanization of Murine Monoclonal Antibody S24B8

Murine monoclonal antibody S24B8 was humanized to reduce its immunogenicity. The humanization scheme adopted a classic framework transplantation strategy (see, Tan P, Mitchell D A, Buss T N, Holmes M A, Anasetti C, Foote J. “Superhumanized” antibodies: reduction of immunogenic potential by complementarity-determining region grafting with human germline sequences: application to an anti-CD28. J Immunol. 2002;169(2):1119-1125, the entire contents of which are incorporated herein by reference). The genes encoding the heavy and light chain variable regions of S24B8 were compared to the human antibody germline genetic sequences in the IMGT database, respectively. Appropriate germline genetic sequences were selected to provide Framework Regions 1 to 3 of the antibody (FR1+FR2+FR3), and an appropriate J region genetic sequence was selected to provide Framework Region 4 (FR4). This template may be selected depending on a variety of factors, such as the relative total length of an antibody, the sizes of the CDRs, the amino acid residues at the junction between the framework region (FR) and the hypervariable region (CDR) of an antibody, and homology of the entire sequence. The selected template may be a mixture of multiple sequences or a common template, in order to maintain an appropriate conformation of the parental complementarity determining regions (CDR) as much as possible. Meanwhile, in order to avoid the protein heterogeneity possibly caused by the deamination site NG in the antibody hypervariable region (CDR), the heavy chain variable region of the humanized antibody was designed for mutations. Finally, three humanized heavy chain variable region mutants S24B8VH-h1(SEQ ID NO: 15), S24B8VH-h2(SEQ ID NO: 16), S24B8VH-h3(SEQ ID NO: 17) and one humanized light chain variable region mutant S24B8VK-h1(SEQ ID NO: 18) were obtained.

### 8.2. Humanization of Murine Monoclonal Antibody S22E2

The humanization of murine monoclonal antibody S22E2 adopted the classic framework transplantation strategy. Referring to Example 8.1, CDR grafting of the light and heavy chain variable regions of S22E2 yielded a humanized heavy chain variable region mutant S22E2VH-h1(SEQ ID NO: 19) and a humanized light chain variable region mutant S22E2VK-h1(SEQ ID NO: 20).

The variable region genes of the antibody were designed and synthesized according to the amino acid sequences of humanized antibodies of S24B8 and S22E2, and cloned into suitable eukaryotic gene expression vectors. The intact antibodies were prepared according to Example 1. Three humanized versions of S24B8 were named S24B8-h1, S24B8-h2, and S24B8-h3. The humanized version of S22E2 was named S22E2-h1.

### 8.3. Affinity Assay of Humanized S24B8 and S22E2

Referring to Example 3.2, the binding affinity between the humanized anti-TT-Hc antibody and TT-Hc was determined by Biacore X100 using a human antibody capture kit (BR-1008-39). The fitting results were shown in Table 2. The heavy chain constant regions of individual humanized antibodies were all of human IgG1 subtype.

TABLE 2
Affinity constant for binding of recombinant
anti-TT-Hc monoclonal antibodies to TT-Hc-His

| | Kₐ | K_{d} | KD |
|---|---|---|---|
| S22E2-IgG1 | 2.042E+5 | 1.872E−4 | 9.163E−10 |
| S22E2-h1-IgG1 | 2.68E+5 | 1.535E−4 | 5.728E−10 |
| S24B8-h1-IgG1 | 7.866E+4 | <1E−5 | — |
| S24B8-h2-IgG1 | 4.447E+4 | 1.713E−5 | 3.852E−10 |
| S24B8-h3-IgG1 | 7.175E+4 | 1.178E−5 | 1.642E−10 |

### Example 9: Preparation of Bispecific Antibodies

Based on the superior neutralizing activity of the combination of anti-tetanus toxin monoclonal antibodies S24B8 and S22E2 over their single treatments, antigen-binding fragments binding to different epitopes of tetanus toxin Hc protein from S24B8-h3 and S22E2-h1 were designed as a Fab and an scFv, respectively, to construct a human IgG1 heterodimer based on the KIH (Knob-Into-Hole) technology. That is, the S24B8-h3-derived antigen-binding fragment in a Fab form was fused to the N-terminus of an Fc segment containing a Knob mutation (FcK, SEQ ID NO: 40), and the S22E2-h1-derived antigen-binding fragment in a scFv form was fused to the N-terminus of an Fc segment containing a Hole mutation (FcH, SEQ ID NO: 41), thereby constructing a bispecific antibody against tetanus toxin (Hc) protein.

The three constructed eukaryotic expression vectors expressing S22E2-h1-scFv-FcH1, S24B8VH-h3+CH-IgG1K and S24B8VK-h1+CK, respectively, were co-transfected into HEK293F cells using liposomes, and the cells were cultured in suspension in a serum-free medium for 3-5 days. The culture supernatant was harvested by centrifugation. The bispecific antibodies in the culture supernatant were purified using a Protein A/G affinity chromatography column (e.g., Mabselect SURE, GE Inc.). The recombinant antibody preservation buffer was then replaced with a PBS buffer (pH 7.0) or other suitable buffers using a desalination column (e.g., Hitrap desaulting, GE Inc.). The desalted protein solution was purified by a size exclusion chromatography (SEC) using Superdex 200 (GE), thereby obtaining bispecific antibody S22E2h1-scFv-FcH1+S24B8h3-IgG1K. If necessary, the antibody samples can be sterilized by filtration and then stored in aliquots at −20° C.

### Example 10: Functional Validation of Bispecific Antibodies

### 10.1. Inhibition of Binding of TT-Hc to Gangliosides

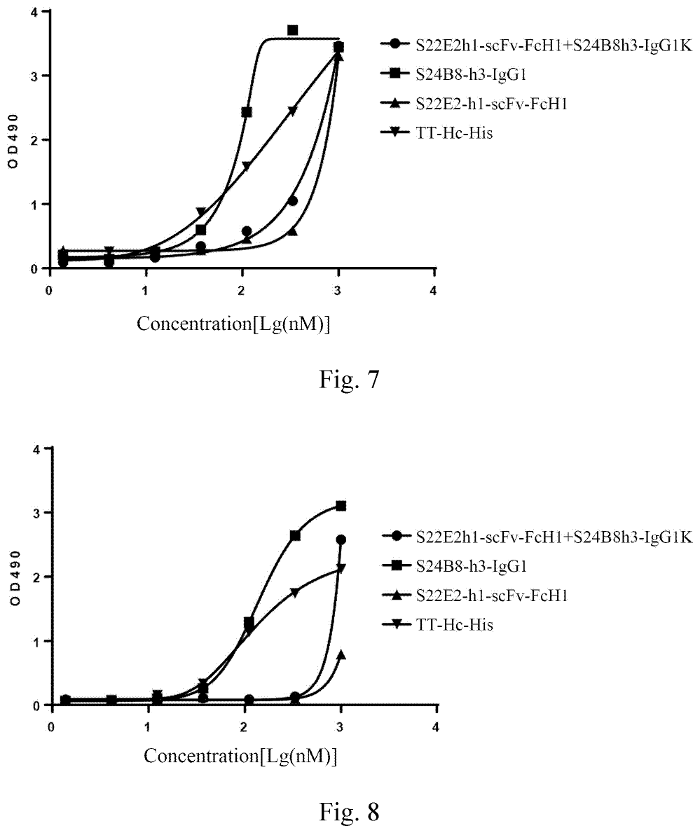

Referring to Example 5, the inhibitory abilities of anti-TT-Hc antibodies on TT-Hc binding to three gangliosides GD3, GT1b and GM1a were determined. ELISA results (FIGS. 6, 7 and 8) showed that monoclonal antibody S22E2-h1-scFv-FcH1 and bispecific antibody S22E2h1-scFv-FcH1+S24B8h3-IgG1K had significant inhibitory effects on binding of TT-Hc to the three gangliosides, and monoclonal antibody S24B8-h3-IgG1 did not affect binding of TT-Hc to the three gangliosides.

### 10.2. Neutralizing Activity

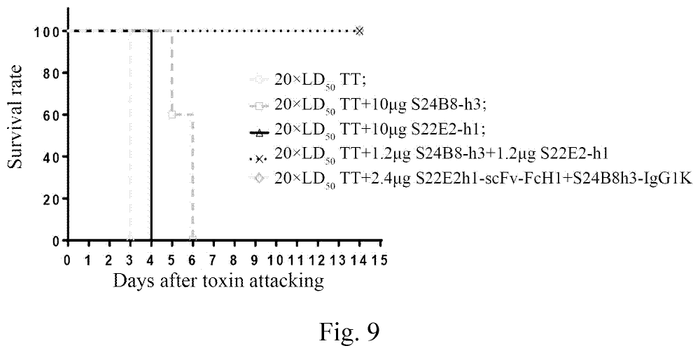

Referring to Example 6, the injection dose of tetanus toxin was 20×LD₅₀/mouse. The injection dose of monoclonal antibody S24B8-h3 or S22E2-h1, when used alone, was 10 μg/mouse. The injection dose of monoclonal antibody S24B8-h3 and S22E2-h1 in combination was 1.2 μg/mouse for each antibody. The injection dose of bispecific antibody S22E2h1-scFv-FcH1+S24B8h3-IgG1K was 2.4 μg/mouse. Balb/c mice injected with 20×LD₅₀ of tetanus toxin were used as a control group. There were a total of 5 groups with 8 mice per group. The tetanus toxin was diluted to 800×LD₅₀/mL with a boric acid buffer (i.e., 20×LD₅₀ per 50 μl of injection after mixing with the antibody in equal volumes). Antibodies S22E2 and S24B8 were diluted to 0.4 mg/mL (or 0.096 mg/mL) with a boric acid buffer (i.e., for the treatment with S22E2 or S24B8, 10 μg of an antibody per 50 μl of injection after mixing with the tetanus toxin in equal volumes; and for the treatment with S24B8 and S22E2 in combination, 1.2 μg of each antibody per 50 μl of injection after mixing the two-antibody mixture (mixed in equal volumes) with the tetanus toxin in equal volumes). Bispecific antibody S22E2h1-scFv-FcH1+S24B8h3-IgG1K was diluted to 0.096 mg/mL with a boric acid buffer (i.e., 2.4 μg of the bispecific antibody per 50 μl of injection after mixing with the tetanus toxin in equal volumes). Each animal was injected with 50 μL of the mixture of the tetanus toxin and an antibody. The experimental results were shown in FIG. 9. All Balb/c mice in the control group died of tetanus within 3 days of the attack, and the Balb/c mice in the combination group of S24B8-h3 and S22E2-h1 and the Balb/c mice in the group of bispecific antibody S22E2h1-scFv-FcH1+S24B8h3-IgG1K all survived by the end of the experimental period. The neutralizing activity of bispecific antibody S22E2h1-scFv-FcH1+S24B8h3-IgG1K was superior to those of the monoclonal antibodies.

### Example 11: Determination of Antibody Titers

The titer of bispecific antibody S22E2h1-scFv-FcH1+S24B8h3-IgG1K was determined based on the method for determining the neutralizing activities of the antibodies in Example 6. The number of international units (IU) of anti-tetanus toxin antibody contained in 1 mg of a test sample was calculated by comparing the test sample and a standard sample. Dilution of tetanus toxin included diluting the tetanus toxin with a boric acid buffer to 40 test doses (L+ 1/10) per milliliter (i.e., 1 test dose (L+ 1/10) per 50 μl of injection after mixing with the antibody in equal volumes). Dilution of a human tetanus immunoglobulin standard included diltuting the human tetanus immunoglobulin standard with a boric acid buffer to 4 IU/mL (i.e., 1/10 IU per 50 μl of injection after mixing with the toxin in equal volumes). Bispecific antibody S22E2h1-scFv-FcH1+S24B8h3-IgG1K was diluted with a boric acid buffer to five concentrations at 15% intervals, i.e., 14.40 μg/mL, 16.80 μg/mL, 20 μg/mL, 23.6 μg/mL and 27.6 μg/mL in sequence (i.e., 0.36 μg, 0.42 μg, 0.50 μg, 0.59 μg and 0.69 μg of the bispecific antibodies per 50 μl of injection after mixing with the toxin in equal volumes). The diluted human tetanus immunoglobulin standard solutions and the solutions of the bispecific antibody at different dilutions were mixed with an equal volume of diluted tetanus toxin, respectively. Immediately after incubation for 1 hour at 37° C., the mixtures were injected to mice with an intramuscular injection volume of 50 μL per mouse. Mice were observed once a day to record incidence or death. The results showed that all Balb/c mice in the control group died of tetanus within 72 hours after the attack, and the highest dose of the bispecific antibody in the group, in which the mice died simultaneously with the mice in the control group, was 0.42 μg, the titer of which was calculated to be 238 IU/mg.

While the present inventions have been described in detail with reference to the general description and specific examples, it will be apparent to those skilled in the art that some modifications and improvements may be made to the present inventions. Accordingly, such modifications and improvements made without departing from the spirit of the inventions fall within the scopes of the inventions as claimed.

### REFERENCE

1. Thwaites C L, Beeching N J, Newton C R (2015) Maternal and neonatal tetanus. Lancet 385:362-370.
2. Yousefi M, Tahmasebi F, Younesi V, Razavi A, Khoshnoodi J, Bayat A A, Abbasi E, Rabbani H, Jeddi-tehrani M, Shokri F (2014a) Characterization of neutralizing monoclonal antibodies directed against tetanus toxin fragment C. J Immunotoxicol 11:28-34.
3. Ashton A C, Li Y, Doussau F, Weller U, Dougan G, Poulain B, Dolly J O (1995) tetanus toxin inhibits neuroexocytosis even when its Zn21-dependent protease activity is removed.
4. Scott N, Qazi O, Wright M J, Fairweather N F, Deonarain M P (2010) Characterisation of a panel of anti-tetanus toxin single-chain Fvs reveals cooperative binding. Mol Immunol 47:1931-1941.
5. Yousefi M, Khosravi-Eghbal R, Reza Mahmoudi A, Jeddi-Tehrani M, Rabbani H, Shokri F (2014b) Comparative in vitro and in vivo assessment of toxin neutralization by anti-tetanus toxin monoclonal antibodies. Hum Vaccin Immunother 10:344-351.
6. Petrusic V, Zivkovic I, Stojanovic M, Stojicevic I, Marinkovic E,Dimitrijevic L (2012) Production, characterization and applications of a tetanus toxin specific monoclonal antibody T-62. Acta Histochem 114:480-486.
7. Lang A B, Cryz S J Jr, Schurch U, Ganss M T, Bruderer U (1993) Immunotherapy with human monoclonal antibodies. Fragment A specificity of polyclonal and monoclonal antibodies is crucial for full protection against tetanus toxin. J Immunol 151:466-472.

1. A bispecific antibody comprising a first antigen-binding fragment and a second antigen-binding fragment that bind to different epitopes of a tetanus toxin, wherein the bispecific antibody has activity of neutralizing the tetanus toxin.

2. The bispecific antibody of claim 1, wherein the first antigen-binding fragment comprises HCDR1 having the amino acid sequence of SYWIY (SEQ ID NO: 1), HCDR2 having the amino acid sequence of EINPTNGFANYNEKFKT (SEQ ID NO: 2) or EINPTAGFANYNEKFKT (SEQ ID NO: 3) or EINPTNAFANYNEKFKT (SEQ ID NO: 4), HCDR3 having the amino acid sequence of HFRFPY (SEQ ID NO: 5), LCDR1 having the amino acid sequence of RASQDIGSSLT (SEQ ID NO: 6), LCDR2 having the amino acid sequence of ATSSLDS (SEQ ID NO: 7) and LCDR3 having the amino acid sequence of LQYASSPYT (SEQ ID NO: 8);
wherein the HCDR and LCDR amino acid sequences are defined according to Kabat.

3. The bispecific antibody of claim 1, wherein the second antigen-binding fragment comprises HCDR1 having the amino acid sequence of DYGVN (SEQ ID NO: 9), HCDR2 having the amino acid sequence of MIWSDGTTDYSSALKS (SEQ ID NO: 10), HCDR3 having the amino acid sequence of VDGYSHYYAMDY (SEQ ID NO: 11), LCDR1 having the amino acid sequence of RASENIYSYLA (SEQ ID NO: 12), LCDR2 having the amino acid sequence of NAKTLAE (SEQ ID NO: 13) and LCDR3 having the amino acid sequence of QHHYGLPFT (SEQ ID NO: 14);
wherein the HCDR and LCDR amino acid sequences are defined according to Kabat.

4. The bispecific antibody of claim 2, wherein
the amino acid sequence of the heavy chain variable region of the first antigen-binding fragment is as set forth in SEQ ID NO: 15, and the amino acid sequence of the light chain variable region of the first antigen-binding fragment is as set forth in SEQ ID NO: 18; or
the amino acid sequence of the heavy chain variable region of the first antigen-binding fragment is set forth in SEQ ID NO: 16, and the amino acid sequence of the light chain variable region of the first antigen-binding fragment is as set forth in SEQ ID NO: 18; or
the amino acid sequence of the heavy chain variable region of the first antigen-binding fragment is as set forth in SEQ ID NO: 17, and the amino acid sequence of the light chain variable region of the first antigen-binding fragment is as set foth in SEQ ID NO: 18.

5. The bispecific antibody of claim 3, wherein the amino acid sequence of the heavy chain variable region of the second antigen-binding fragment is as set forth in SEQ ID NO: 19, and the amino acid sequence of the light chain variable region of the second antigen-binding fragment is as set forth in SEQ ID NO: 20.

6. The bispecific antibody of claim 1, wherein the forms of the first antigen-binding fragment and the second antigen-binding fragment are independently selected from a single-chain fragment variable (scFv) or a Fab fragment.

7. A monoclonal antibody that binds to a tetanus toxin, comprising:
HCDR1 having the amino acid sequence of SYWIY (SEQ ID NO: 1),
HCDR2 having the amino acid sequence of EINPTNGFANYNEKFKT (SEQ ID NO: 2) or
EINPTAGFANYNEKFKT (SEQ ID NO: 3) or EINPTNAFANYNEKFKT (SEQ ID NO: 4),
HCDR3 having the amino acid sequence of HFRFPY (SEQ ID NO: 5),
LCDR1 having the amino acid sequence of RASQDIGSSLT (SEQ ID NO: 6),
LCDR2 having the amino acid sequence of ATSSLDS (SEQ ID NO: 7), and
LCDR3 having the amino acid sequence of LQYASSPYT (SEQ ID NO: 8);
wherein the HCDR and LCDR amino acid sequences are defined according to Kabat.

8. A monoclonal antibody that binds to a tetanus toxin, comprising:
HCDR1 having the amino acid sequence of DYGVN (SEQ ID NO: 9),
HCDR2 having the amino acid sequence of MIWSDGTTDYSSALKS (SEQ ID NO: 10), HCDR3 having the amino acid sequence of VDGYSHYYAMDY (SEQ ID NO: 11), LCDR1 having the amino acid sequence of RASENIYSYLA (SEQ ID NO: 12),
LCDR2 having the amino acid sequence of NAKTLAE (SEQ ID NO: 13), and
LCDR3 having the amino acid sequence of QHHYGLPFT (SEQ ID NO: 14);
wherein the HCDR and LCDR amino acid sequences are defined according to Kabat.

9. A pharmaceutical composition comprising the bispecific antibody of claim 1, and a pharmaceutically acceptable excipient, diluent or carrier.

10. The pharmaceutical composition of claim 9, which is for use in the prevention or treatment of tetanus.

11. (canceled)

12. A method of preventing or treating tetanus comprising administering to a subject in need thereof the bispecific antibody of claim 1.

13. The bispecific antibody of claim 1, wherein the first antigen-binding fragment is a Fab fragment, and the second antigen-binding fragment is a single-chain fragment variable (scFv).

14. The bispecific antibody of claim 1, wherein the bispecific antibody comprises the amino acid sequence set forth in one of SEQ ID NOs: 21, 22 and 23 and the amino acid sequence set forth in SEQ ID NO: 24.

15. The bispecific antibody of claim 1, wherein the bispecific antibody comprises the amino acid sequence set forth in SEQ ID NO: 25.

16. The monoclonal antibody of claim 7, wherein the monoclonal antibody comprises:
a heavy chain variable region amino acid sequence set forth in SEQ ID NO: 15, and a light chain variable region amino acid sequence set forth in SEQ ID NO: 18; or
a heavy chain variable region amino acid sequence set forth in SEQ ID NO: 16, and a light chain variable region amino acid sequence set forth in SEQ ID NO: 18; or
a heavy chain variable region amino acid sequence set forth in SEQ ID NO: 17, and a light chain variable region amino acid sequence set forth in SEQ ID NO: 18.

17. The monoclonal antibody of claim 8, wherein the monoclonal antibody comprises a heavy chain variable region amino acid sequence set forth in SEQ ID NO: 19, and a light chain variable region amino acid sequence set forth in SEQ ID NO: 20.


SEQUENCE LISTING (41 sequences)

SEQ ID NO: 1 - protein, 5 aa, Artificial Sequence
  Synthetic Polypeptide
SYWIY

SEQ ID NO: 2 - protein, 17 aa, Artificial Sequence
  Synthetic Polypeptide
EINPTNGFANYNEKFKT

SEQ ID NO: 3 - protein, 17 aa, Artificial Sequence
  Synthetic Polypeptide
EINPTAGFANYNEKFKT

SEQ ID NO: 4 - protein, 17 aa, Artificial Sequence
  Synthetic Polypeptide
EINPTNAFANYNEKFKT

SEQ ID NO: 5 - protein, 6 aa, Artificial Sequence
  Synthetic Polypeptide
HFRFPY

SEQ ID NO: 6 - protein, 11 aa, Artificial Sequence
  Synthetic Polypeptide
RASQDIGSSLT

SEQ ID NO: 7 - protein, 7 aa, Artificial Sequence
  Synthetic Polypeptide
ATSSLDS

SEQ ID NO: 8 - protein, 9 aa, Artificial Sequence
  Synthetic Polypeptide
LQYASSPYT

SEQ ID NO: 9 - protein, 5 aa, Artificial Sequence
  Synthetic Polypeptide
DYGVN

SEQ ID NO: 10 - protein, 16 aa, Artificial Sequence
  Synthetic Polypeptide
MIWSDGTTDYSSALKS

SEQ ID NO: 11 - protein, 12 aa, Artificial Sequence
  Synthetic Polypeptide
VDGYSHYYAMDY

SEQ ID NO: 12 - protein, 11 aa, Artificial Sequence
  Synthetic Polypeptide
RASENIYSYLA

SEQ ID NO: 13 - protein, 7 aa, Artificial Sequence
  Synthetic Polypeptide
NAKTLAE

SEQ ID NO: 14 - protein, 9 aa, Artificial Sequence
  Synthetic Polypeptide
QHHYGLPFT

SEQ ID NO: 15 - protein, 115 aa, Artificial Sequence
  Synthetic Polypeptide
QVQLVQSGAEVKKPGSSVKVSCKASGYTFTSYWIYWVRQAPGQGLEWIGEINPTNGFANYNEKFKTKATITVDKSTSTAYMELSSLRSEDTAVYYCAIHFRFPYWGQGTMVTVSS

SEQ ID NO: 16 - protein, 115 aa, Artificial Sequence
  Synthetic Polypeptide
QVQLVQSGAEVKKPGSSVKVSCKASGYTFTSYWIYWVRQAPGQGLEWIGEINPTAGFANYNEKFKTKATITVDKSTSTAYMELSSLRSEDTAVYYCAIHFRFPYWGQGTMVTVSS

SEQ ID NO: 17 - protein, 115 aa, Artificial Sequence
  Synthetic Polypeptide
QVQLVQSGAEVKKPGSSVKVSCKASGYTFTSYWIYWVRQAPGQGLEWIGEINPTNAFANYNEKFKTKATITVDKSTSTAYMELSSLRSEDTAVYYCAIHFRFPYWGQGTMVTVSS

SEQ ID NO: 18 - protein, 107 aa, Artificial Sequence
  Synthetic Polypeptide
DIQMTQSPSSLSASVGDRVTITCRASQDIGSSLTWLQQKPGKAPKRLIYATSSLDSGVPSRFSGSGSGTDYTLTISSLQPEDFATYYCLQYASSPYTFGQGTKLEIK

SEQ ID NO: 19 - protein, 120 aa, Artificial Sequence
  Synthetic Polypeptide
QVTLKESGPVLVKPTETLTLTCTVSGFSLTDYGVNWIRQPPGKALEWLGMIWSDGTTDYSSALKSRLTISKDTSKSQVVLTMTNMDPVDTATYYCARVDGYSHYYAMDYWGQGTLVTVSS

SEQ ID NO: 20 - protein, 107 aa, Artificial Sequence
  Synthetic Polypeptide
DIQMTQSPSSLSASVGDRVTITCRASENIYSYLAWYQQKQGKAPQLLIYNAKTLAEGVSSRFSGSGSGTQFTLTISSLQPEDFATYYCQHHYGLPFTFGQGTKLEIK

SEQ ID NO: 21 - protein, 445 aa, Artificial Sequence
  Synthetic Polypeptide
QVQLVQSGAEVKKPGSSVKVSCKASGYTFTSYWIYWVRQAPGQGLEWIGEINPTNGFANYNEKFKTKATITVDKSTSTAYMELSSLRSEDTAVYYCAIHFRFPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 445 aa, Artificial Sequence
  Synthetic Polypeptide
QVQLVQSGAEVKKPGSSVKVSCKASGYTFTSYWIYWVRQAPGQGLEWIGEINPTAGFANYNEKFKTKATITVDKSTSTAYMELSSLRSEDTAVYYCAIHFRFPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 445 aa, Artificial Sequence
  Synthetic Polypeptide
QVQLVQSGAEVKKPGSSVKVSCKASGYTFTSYWIYWVRQAPGQGLEWIGEINPTNAFANYNEKFKTKATITVDKSTSTAYMELSSLRSEDTAVYYCAIHFRFPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Artificial Sequence
  Synthetic Polypeptide
DIQMTQSPSSLSASVGDRVTITCRASQDIGSSLTWLQQKPGKAPKRLIYATSSLDSGVPSRFSGSGSGTDYTLTISSLQPEDFATYYCLQYASSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 474 aa, Artificial Sequence
  Synthetic Polypeptide
QVTLKESGPVLVKPTETLTLTCTVSGFSLTDYGVNWIRQPPGKALEWLGMIWSDGTTDYSSALKSRLTISKDTSKSQVVLTMTNMDPVDTATYYCARVDGYSHYYAMDYWGQGTLVTVSSGGGGSGGGGSGGGGSDIQMTQSPSSLSASVGDRVTITCRASENIYSYLAWYQQKQGKAPQLLIYNAKTLAEGVSSRFSGSGSGTQFTLTISSLQPEDFATYYCQHHYGLPFTFGQGTKLEIKEPKSSDKTHTCPPCPAPELLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSPGK

SEQ ID NO: 26 - protein, 451 aa, Artificial Sequence
  Synthetic Polypeptide
KNLDAWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 27 - protein, 6 aa, Artificial Sequence
  Synthetic Polypeptide
HHHHHH

SEQ ID NO: 28 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 30 - protein, 330 aa, Artificial Sequence
  Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 330 aa, Artificial Sequence
  Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 330 aa, Artificial Sequence
  Synthetic Polypeptide
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 33 - protein, 330 aa, Artificial Sequence
  Synthetic Polypeptide
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 34 - protein, 107 aa, Homo sapiens [sequence omitted]

SEQ ID NO: 35 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - protein, 106 aa, Mus musculus
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 38 - DNA, 19 bp, Artificial Sequence
  Synthetic Polynucleotide
tgcatttgaactccttgcc

SEQ ID NO: 39 - DNA, 20 bp, Artificial Sequence
  Synthetic Polynucleotide
ccatcaatcttccacttgac

SEQ ID NO: 40 - protein, 232 aa, Artificial Sequence
  Synthetic Polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 232 aa, Artificial Sequence
  Synthetic Polypeptide
EPKSSDKTHTCPPCPAPELLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSPGK